(19) United States
(12) Patent Application Publication
(10) Pub. No.: US 2024/0182516 A1
(43) Pub. Date: Jun. 6, 2024

(54) METHODS FOR CONVENIENT ASYMMETRIC SYNTHESIS OF C9-SUBSTITUTED STEROID-LIKE COMPOUNDS

(71) Applicant: TRUSTEES OF DARTMOUTH COLLEGE, Hanover, NH (US)

(72) Inventor: Glenn C. MICALIZIO, Norwich, VT (US)

(21) Appl. No.: 18/551,249

(22) PCT Filed: Mar. 17, 2022
(86) PCT No.: PCT/US2022/071200

§ 371 Date: Sep. 19, 2023

Related U.S. Application Data
(60) Provisional application No. 63/163,469, filed on Mar. 19, 2021.

(51) Int. Cl. C07J 75/00; A61K 31/566; C07J 1/00

(52) U.S. Cl. CPC C07J 75/005; A61K 31/566; C07J 1/00

(57) ABSTRACT

The present disclosure relates to stereodefined polycyclic (e.g., tetracyclic) compounds that contain quaternary centers at one or multiple ring fusions, synthetic methods for preparing such compounds. In one aspect, the present disclosure relates to preparing a C9-alpha-substituted or a C9-beta-substituted compound (steroid numbering), the method comprising the steps of providing a tertiary allylic substrate and performing a regio- and stereoselective cyclization reaction to form a C9-C10 bond and to set a quaternary center at C9.

### CROSS REFERENCE TO RELATED APPLICATIONS

This patent application claims priority to U.S. Provisional Patent Application No. 63/163,469, filed on Mar. 19, 2021, the entire contents of which are fully incorporated herein by reference.

### FEDERALLY SPONSORED RESEARCH OR DEVELOPMENT

This invention was made with government support under R01 GM080266 awarded by the National Institutes of Health and R35 GM134725 awarded by the National Institutes of Health. The government has certain rights in the invention.

### FIELD OF THE INVENTION

The present disclosure provides concise synthetic methods for accessing stereodefined carbocycles that contain quaternary centers at one or multiple ring fusions, compounds accessible by such synthetic methods, and methods of using such compounds to treat a disease, including diseases mediated by nuclear hormone receptors such as estrogen receptor beta (ERβ), androgen receptor (AR), and glucocorticoid receptor (GR).

### BACKGROUND OF THE INVENTION

Steroids and tetracyclic terpenoids (more broadly), including unnatural variants, have had a transformative impact on medicine and society, playing vital roles as oral contraceptives, treatments for cancer (including anti-angiogenic agents), heart failure, inflammation, pain, and traumatic brain injuries, among others, and natural molecules in this class have served as important chemical precursors in drug discovery and development. Despite this rich history, substantial barriers persist that greatly limit the types of synthetic compositions of matter in this broad area of chemical space that can efficiently be prepared and explored as potential medicines and biological tools/probes

Many presently available synthetic and semisynthetic routes to molecules in this class are often complex, inefficient, and/or wholly incapable of producing advantageous collections (i.e., libraries) of highly oxygenated/functionalized target compositions necessary for advancement through modern drug development. Indeed, efficient de novo synthesis of “steroidal” systems, or tetracyclic terpenoid-inspired compositions of matter, remains a challenging problem in chemistry.

All of the more than 100 FDA-approved drugs in this area of chemical space are of the natural enantiomer (specific reference being made to the absolute stereochemistry at C13 of the steroidal skeleton)—a fact that is certainly influenced by the manner in which such compounds are prepared. In fact, it is typical that medicinal agents in this class are synthesized from naturally occurring steroids. As compared to steroidal compositions of matter that have the natural absolute stereochemistry at C13 (“nat-steroids”), synthetic ent-steroidal compounds (defined by an unnatural absolute stereochemistry) have complementary three-dimensional structures while offering similar “drug-like” physical properties. As a result, synthetic ent-steroids are privileged natural product-inspired scaffolds of great potential therapeutic relevance, and are distinct compositions in comparison to their natural isomers. (See, Akwa, Y., et al., Proc. Natl. Acad. Sci. U.S.A., 98, 14033-14037 [2001]; Green, P. S., et al., Endocrinology, 142, 400-406 [2001]; Biellmann, J. F., Chem. Rev., 103, 2019-2033 [2003]; Covey, D. F., Steroids, 74 (7):577-585 [2009]; and Petit, G. H., et al., Eur. Neuropsychopharmacol., 21, 211-215 [2011]). However, investigations of the unnatural enantiomers of steroid-inspired compounds have been hampered in the past due to the great difficulty associated with preparing/accessing such compositions of matter. While semisynthetic routes to nat-steroids (i.e., those beginning with a readily available steroid or related natural product) have been incredibly powerful, such preparative methods are not suitable for producing non-naturally occurring ent-steroids because the starting material possesses a mirror image backbone inherent to natural molecules in the class. In summary, ent-steroids are an important class of privileged pharmaceutical drug-like molecules that presently cannot be fully leveraged in biological and pharmaceutical research efforts because these molecules are not readily available from natural sources and existing chemical synthesis pathways are inefficient and not flexible enough to produce diverse collections of such molecules suitable for drug discovery and development.

A practical method for efficient and stereospecific production of ent-steroids, as well as other unnatural stereoisomers and simply unique compositions of matter within the broad class of tetracyclic terpenoids, would enable scientists and physicians to better exploit the as yet untapped potential of new molecules within this pharmaceutically-privileged class (including ent-steroids) as useful tools and therapeutics. In fact, even within the nat-steroid family of potential medicines, the current state-of-the-art that relies heavily on semisynthesis (where synthesis proceeds from a readily available natural product) comes with significant limitations based on the structure of the abundant and readily available natural material (e.g., level of unsaturation, density of oxygenation, and degree of substitution of the starting material). As such, even with state-of-the-art approaches, vast regions of privileged chemical space for medicinal science remain difficult to explore. Accordingly, what is needed are efficient and step-economical (i.e., concise), flexible, convergent, and enantiospecific methods of synthesizing synthetic nat- and/or ent-steroids having varying stereochemistry and substitution, and/or functionality that facilitates subsequent molecular perturbation processes (i.e., manipulation of functionality in each ring of the characteristic tetracyclic nucleus) at research and/or production scale.

One recent advance in this area is the establishment of a synthetic route from epichlorohydrin to steroidal tetracycles bearing a quaternary center at C9. See, e.g., Kim et al., Nat. Comm. 10, 2448 (2019); WO2020051329. This synthetic route comprises a modern metallacycle-mediated annulative cross-coupling, a C9-C10 bond-forming process (e.g., through a double-asymmetric Friedel-Crafts cyclization or an intramolecular Heck reaction), and, optionally, an oxidative rearrangement reaction. This platform allows for construction of central motifs of tetracyclic terpenoid carbocyclic backbones in just a handful of steps from an inexpensive and readily available chiral starting material (epichlorohydrin). Nevertheless, there is a need for additional synthetic routes that address certain limitations of existing technology. For example, incorporation of molecular substituents at C17 is cumbersome with existing technology, requiring, for example, modification of the starting material.

### SUMMARY OF THE INVENTION

The present disclosure relates to methods for producing stereodefined polycyclic ring compounds, including enantiodefined systems, through unique intermediates, synthesis strategies, and chemical reactions. More particularly, the present disclosure provides synthetic methods for producing a natural product-inspired complex polycyclic tetracycle, including, but not limited to, a compound having a “C19 steroidal scaffold.” As used herein, the term “C19 steroidal scaffold” includes not only steroids, and compounds that could be defined as steroidal, that have 19 carbon atoms, but also includes compounds having additional carbon atoms, including, but not limited to C20, C21, C22, C23, C24, C25, C26, C27, C28, C29, C30, or C31 compounds.

In particular, the present disclosure relates to a concise approach to the assembly of fused carbocyclic structures through a novel sequence of chemical transformations that includes a cyclization reaction to forge the “steroidal” C9-C10 bond, establishing a quaternary center at C9. In certain embodiments, the methods involve a regio- and stereoselective cyclization reaction to form a C9-C10 bond and to set a quaternary center at C9, wherein the reaction is understood to likely proceed through a cationic intermediate to yield a C9-alpha-substituted steroid-like compound or a C9-beta-substituted steroid-like compound. The method is selective for formation of the anti-isomer. As a result, the group attached to C9 projects on the opposite face of the tetracycle as the preexisting group at C13.

In certain embodiments, the methods further involve an oxidative dearomatization and group-selective Wagner-Meerwein rearrangement marked by a 1,2-alkyl shift from C9 to C10.

The present disclosure also relates to steroidal compounds, including molecules with natural (“nat-”) absolute stereochemistry and compounds with unnatural (“ent-”) absolute stereochemistry, as well as synthetic variants based on such skeletons. In certain embodiments, the compounds have a C19 steroidal scaffold. In other embodiments, compounds having a C19 steroidal scaffold enable access to further compounds based on, or derived from, the C19 scaffold, such as non-natural antipodes of synthetic agents related to natural terpenoids (i.e., steroids, limonoids, bufadienolides, etc.).

The present disclosure also relates to steroidal compounds having quaternary centers at C9 and C13. In certain embodiments, the compounds, with reference to the quaternary centers at C9 and C13, are anti-isomers (the quaternary center at C9 projects a substituent on the opposite face of the tetracycle as the substituent at C13). For example, the compounds may be C9-α-alkyl (or C9-α-aryl) and C13-β-alkyl (or C13-β-aryl) or, alternatively, C9-β-alkyl (or C9-β-aryl) and C13-α-alkyl (or C13-α-aryl), wherein any alkyl or aryl substituent is optionally substituted.

The present disclosure also relates to steroidal compounds having quaternary centers at C10 and C13. In certain embodiments, the compounds, with reference to the quaternary centers at C10 and C13, are anti-isomers (the quaternary center at C10 projects a substituent on the opposite face of the tetracycle as the substituent at C13). For example, the compounds may be C10-α-alkyl (or C10-α-aryl) and C13-β-alkyl (or C13-β-aryl) or, alternatively, C10-β-alkyl (or C10-β-aryl) and C13-α-alkyl (or C13-α-aryl), wherein any alkyl or aryl substituent is optionally substituted.

The present disclosure also relates to the use of such compounds as biologically active (e.g., therapeutic) components in, for example, pharmaceutical compositions and/or directly as human and/or animal therapeutics and medicines. In certain embodiments, the compounds modulate nuclear hormone receptors.

The compounds, pharmaceutical compositions comprising the compounds, and methods for treating or preventing conditions, disorders, or diseases by administering the compounds are further described herein.

These and other objects of the invention are described in the following paragraphs. These objects should not be deemed to narrow the scope of the invention.

### BRIEF DESCRIPTION OF THE DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art.

FIG. 1 shows ¹H NMR and ¹³C NMR spectra for cyclic enone 1, cyclization substrate 1, and steroid 1.

### DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

In certain aspects, the present disclosure relates to compounds (and methods of making such compounds, compositions comprising such compounds, and methods of using such compounds) comprising a generic tetracyclic steroidal (A, B, C, D) ring structure, as follows:

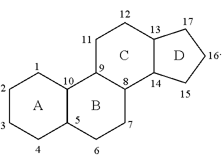

Each carbon ring atom of the generic tetracyclic steroidal ring structure is numbered according to the numbering convention for steroid molecules, which is known in the art and has been explained, for example, in Moss G. P. Nomenclature of Steroids, Pure & Appl. Chem., 61 (10) 1783-1822 (1989), which is hereby incorporated by reference in its entirety. Particular carbon atoms in the structures of the various disclosed formulas are referred to herein by “C” number, for example, C1, C2, C3, C9, C10, C13, etc. The number is reserved to a particular position in that parent skeletal structure whether that position is occupied by a carbon atom or not.

More particularly, the present disclosure relates to compounds (and methods of making such compounds, compositions comprising such compounds, and methods of using such compounds) comprising a generic C19 steroidal core skeleton of Formula (I) or Formula (II), where additional substitution about these base structures is intended to be within the scope of the invention:

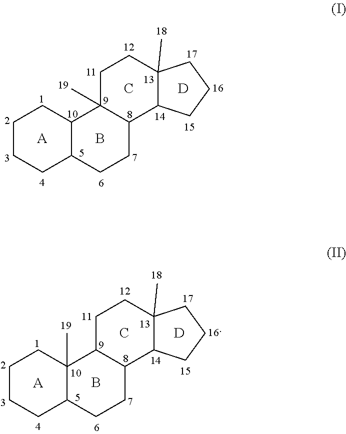

In one aspect, this disclosure provides a composition comprising a collection of synthetic stereoisomers having a chemical structure including a C19 steroidal core skeleton of Formula (I), said C19 steroidal core skeleton having a quaternary center at each of carbon C9 and carbon C13, including stereoisomeric variation among the collection of synthetic stereoisomers; wherein the composition comprises greater than about 70%, alternatively greater than about 75%, alternatively greater than about 80%, alternatively greater than about 85%, alternatively greater than about 90%, or alternatively greater than about 95% of a single C9/C13 stereoisomer relative to other C9/C13 stereoisomers.

In certain embodiments, the single C9/C13 stereoisomer is a diastereomer of a natural compound. In certain embodiments, the single C9/C13 stereoisomer is an enantiomer of a natural compound.

In certain embodiments, the single C9/C13 stereoisomer has a chemical structure including Formula (I-1.1) or Formula (I-1.2):

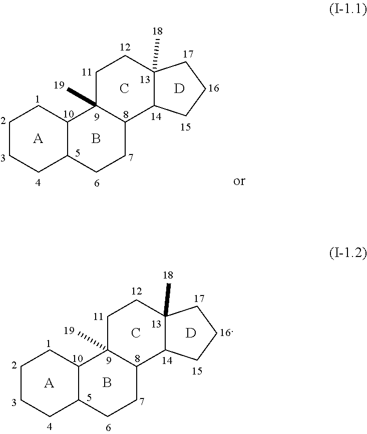

The C19 steroidal core skeleton depicted above in Formula (1-1.1) or Formula (I-1.2) encompasses, inter alia, a steroidal core skeleton, such as Formula (I-2.1) or Formula (1-2.2), respectively:

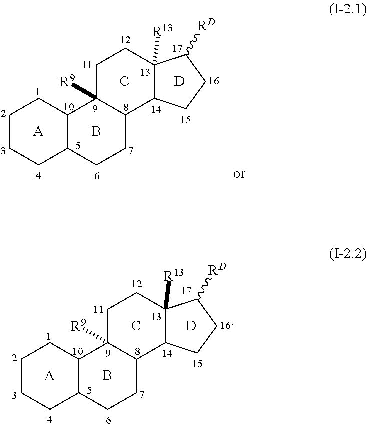

In another aspect, this disclosure provides a composition comprising a collection of synthetic stereoisomers having a chemical structure including a C19 steroidal core skeleton of Formula (II), said C19 steroidal core skeleton having a quaternary center at each of carbon C10 and carbon C13, including stereoisomeric variation among the collection of synthetic stereoisomers; wherein the composition comprises greater than about 70%, alternatively greater than about 75%, alternatively greater than about 80%, alternatively greater than about 85%, alternatively greater than about 90%, or alternatively greater than about 95% of a single C10/C13 stereoisomer relative to other C10/C13 stereoisomers.

In certain embodiments, the single C10/C13 stereoisomer is a diastereomer of a natural compound. In certain embodiments, the single C10/C13 stereoisomer is an enantiomer of a natural compound.

In certain embodiments, the single C10/C13 stereoisomer has a chemical structure including Formula (II-1.1) or Formula (II-1.2):

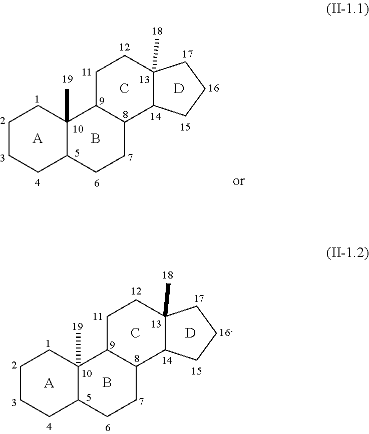

The C19 steroidal core skeleton depicted above in Formula (II-1.1) or Formula (II-1.2) encompasses, inter alia, a steroidal core skeleton, such as Formula (II-2.1) or Formula (II-2.2), respectively:

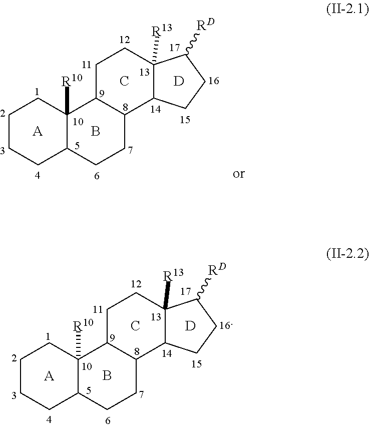

The numbering convention throughout the present disclosure is in accordance with numbered structures above.

In reference to the generic tetracyclic steroidal (A, B, C, D) ring structure and the generic C19 and C20 steroidal core skeletons, it will be well appreciated that in view of the disclosure contained herein as well as the teachings in the relevant fields of art, the compounds, compositions, and methods of the present disclosure are not limited to any particular respective constituent (R) group(s) at the various numbered carbon atoms. For example, an R group may be hydrogen, a C₁₋₁₀-aliphatic group, a C₆₋₁₀ aromatic group, carboxylic acid, carboxylic acid ester, hydroxyl, or halogen. Moreover, it will be well appreciated that in view of the disclosure contained herein as well as the teachings in the relevant fields of art, the compounds, compositions, and methods of the present disclosure may comprise ones in which the A ring can be saturated, partially unsaturated, or completely unsaturated (i.e., aromatic); likewise, the B ring can be saturated, partially unsaturated, or completely unsaturated; the C ring and the D ring can independently be saturated or partially unsaturated.

In an exemplary embodiment, with reference to Formula (I-2.1) or Formula (1-2.2), each of C1, C2, C3, C4, C5, C6, C7, C8, C10, C11, C12, C14, C15, and C16 is independently substituted with hydrogen, C₁₋₁₀-alkyl, C₂₋₂₀-alkenyl, C₂₋₂₀-alkynyl, C₁₋₁₀-haloalkyl, halogen, oxo, hydroxy, C₁₋₆-alkoxy, —O—C₁₋₁₀-alkyl, —O—C₂₋₁₀-alkenyl, —O—C₂₋₁₀-alkynyl, —O—C₁₋₁₀-haloalkyl, —O—C₆₋₁₀-aryl, —O-5- to 10-membered heteroaryl, —OC(O)—C₁₋₁₀-alkyl, —OC(O)—C₆₋₁₀-aryl, —OC(O)-5- to 10-membered heteroaryl, C₆₋₁₀-aryl, or 5- to 10-membered heteroaryl and R⁹, R¹³, and R^{D} are defined herein.

In an exemplary embodiment, with reference to Formula (II-2.1) or Formula (II-2.2), each of C1, C2, C3, C4, C5, C6, C7, C8, C9, C11, C12, C14, C15, and C16 is independently substituted with hydrogen, C₁₋₁₀-alkyl, C₂₋₁₀-alkenyl, C₂₋₁₀-alkynyl, C₁₋₁₀-haloalkyl, halogen, oxo, hydroxy, C₁₋₆-alkoxy, —O—C₁₋₁₀-alkyl, —O—C₂₋₁₀-alkenyl, —O—C₂₋₁₀-alkynyl, —O—C₁₋₁₀-haloalkyl, —O—C₆₋₁₀-aryl, —O-5- to 10-membered heteroaryl, —OC(O)—C₁₋₁₀-alkyl, —OC(O)—C₆₋₁₀-aryl, —OC(O)-5- to 10-membered heteroaryl, C₆₋₁₀-aryl, or 5- to 10-membered heteroaryl and R¹⁰, R¹³, and R^{D} are defined herein.

### A. DEFINITIONS

As used in the specification and the appended claims, unless specified to the contrary, the following terms have the meaning indicated:

The term “about” as used herein, means approximately, and in most cases within 10% of the stated value.

The term “aliphatic” as used herein, includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. In some embodiments, one or more units (e.g., methylene units) of an aliphatic may be replaced with —O—, —NR^{x}—, —C(O)—, or —S(O)n—, where R^{x} is hydrogen or C₁₋₆-alkyl and n is 0, 1, or 2. As will be appreciated by one of ordinary skill in the art, “aliphatic” is intended herein to include alkyl, alkenyl, alkynyl, cycloalkyl, and cycloalkenyl moieties.

The term “pharmaceutically acceptable” is used adjectivally to mean that the modified noun is appropriate for use as a pharmaceutical product for human use or as a part of a pharmaceutical product for human use.

The term “prodrug” refers to a compound that can be readily converted (e.g., metabolized) in vivo to yield a parent compound. Prodrugs include, but are not limited to, compounds having a substituent, such an ester moiety, attached to a hydroxy group at C3 (steroid numbering), which yield a parent compound having a phenolic A ring upon in vivo conversion. Suitable C3 substituents are identified in US2007/0015740 A1, which is herein incorporated by reference in its entirety. Exemplary ester moieties include, but are not limited to, an alkyl ester (e.g., —O—C₁₋₆-alkyl), a carbonate ester (e.g., —O—C(O)—O—C₁₋₁₀-alkyl), a carbamate ester (e.g., —O—C(O)—NR^{Z1}R^{Z2}), and a sulfamate ester (e.g., —O—S(O)₂NR^{Z1}R^{Z2}). Additionally or alternatively, prodrugs may have a substituent, such as an optionally substituted 5- to 10-membered heteroaryl, attached to carbon C17 (steroid numbering), such as those identified in US2014/0371181 A1, which is herein incorporated by reference in its entirety. Prodrugs also include, but are not limited to, di-steroidal prodrugs such as those disclosed in U.S. Pat. No. 7,067,505, which is herein incorporated by reference in its entirety.

The terms “treat”, “treating” and “treatment” refer to a method of alleviating or abrogating a condition, disorder, or disease and/or the attendant symptoms thereof.

### B. SYNTHETIC METHODS AND INTERMEDIATE COMPOUNDS

In one aspect, the present disclosure provides a method for preparing a C9-alpha-substituted or a C9-beta-substituted steroid-like compound (steroid numbering), the method comprising a step of performing a regio- and stereoselective cyclization reaction to form a C9-C10 bond and to set a quaternary center at C9. The reaction is understood to likely proceed through a cationic intermediate to yield the C9-alpha-substituted steroid-like compound or the C9-beta-substituted steroid-like compound. In certain embodiments, the regio- and stereoselective cyclization reaction is mediated by a protic or Lewis acid, such as SnCl₄.

In certain embodiments, the method further comprises an oxidative dearomatization reaction that is terminated by a suprafacial 1,2-shift, in some cases also including loss of a proton, to deliver a fused polycyclic system containing a quaternary center at the ring fusion (at the steroidal C10 carbon).

The following general Schemes 1-6 are representative of particular embodiments of the method:

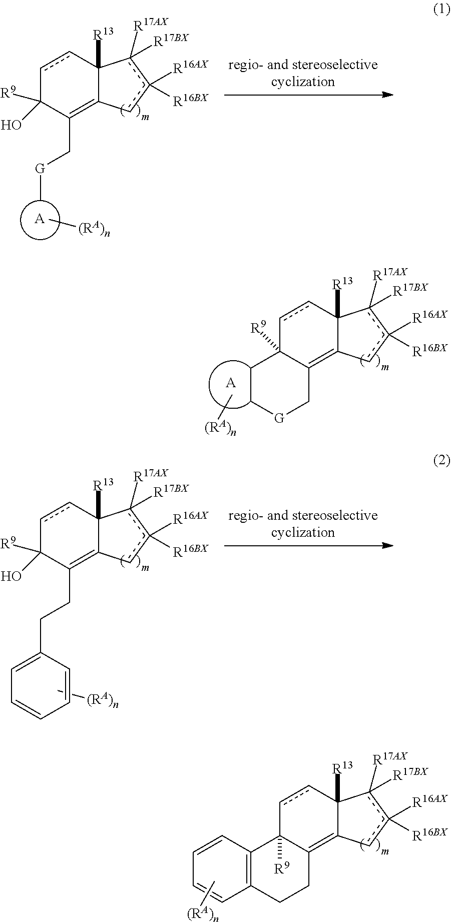

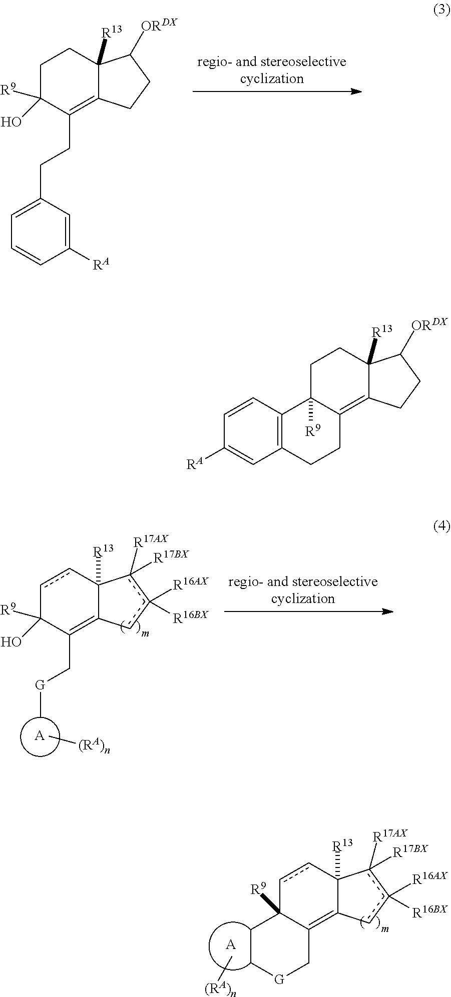

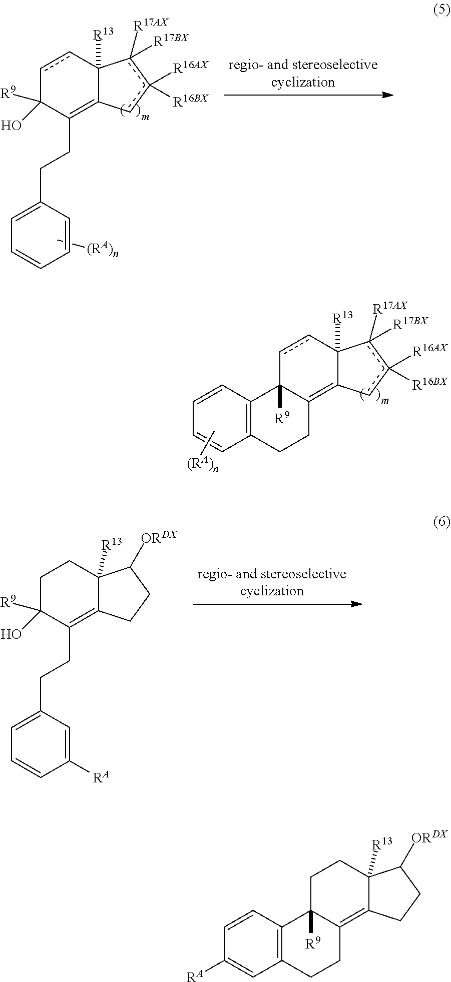

wherein n, m, R_{A}, G, R⁹, R¹³, R^{16AX}, R^{16BX}, R^{17AX}, R^{17BX}, and R^{DX} may be any substituent described herein. In certain embodiments, the reaction is mediated by a protic or Lewis acid, such as SnCl₄.

In these general Schemes 1-6, the carbon atom bearing the R⁹ and —OH substituents is the C9 carbon atom and the A ring and/or phenyl ring bearing the RA substituent contains the C10 carbon atom. The cyclization reaction forms a C9-C10 bond and sets a quaternary center at C9.

In certain embodiments, R^{A} is —O—C₁₋₆-alkyl, such as —OMe. In certain embodiments, one of R⁹ and R¹³ are both independently C₁₋₆-alkyl, such as methyl. In certain embodiments, R^{DX} is TBS.

In certain embodiments, the method (e.g., including an appropriate cyclization step as described herein) provides high regioselectivity (i.e., rs>10:1 and even ≥20:1) and high stereoselectivity (i.e., ds>10:1 and even ≥20:1).

In some such embodiments, the method produces a composition having at least 80%, at least 85%, at least 90%, at least 95%, at least 97%, or at least 99% diastereomeric purity. In some such embodiments, the method does not include a chiral purification step (e.g., resolution by crystallization or chromatography). In some such embodiments, the method produces a composition having at least 85% of one diastereomer and not more than 15% of any other diastereomer. In some such embodiments, the method produces a composition having at least 90% of one diastereomer and not more than 10% of any other diastereomer. In some such embodiments, the method produces a composition having at least 95% of one diastereomer and not more than 5% of any other diastereomer. In some such embodiments, the method produces a composition having at least 97% of one diastereomer and not more than 3% of any other diastereomer. In some such embodiments, the method produces a composition having at least 99% of one diastereomer and not more than 1% of any other diastereomer. In certain embodiments, a chiral purification step may be employed to access enantioenriched/pure products as needed based on the optical purity of the chiral intermediates employed. In certain embodiments, the method employs an optically pure starting material. Thus, in certain embodiments, the desired purity can be achieved without the use of a chiral purification step.

In certain embodiments, the method proceeds with an enantioenriched cyclization substrate (for example, where the substituent at C13 is beta) and produces a composition having at least 80%, at least 85%, at least 90%, at least 95%, at least 97%, or at least 99% enantiomeric purity. In some such embodiments, the method does not include a chiral purification step (e.g., resolution by crystallization or chromatography). In some such embodiments, the method produces a composition having at least 85% of one enantiomer (e.g., where the substituent at C9 is alpha and the substituent at C13 is beta) and produces a composition having at least 80%, at least 85%, at least 90%, at least 95%, at least and not more than 15% of the other enantiomer (e.g., where the substituent at C9 is beta and the substituent at C13 is alpha) without employing a chiral purification step. In some such embodiments, the method produces a composition having at least 90% of one enantiomer and not more than 10% of the other enantiomer without employing a chiral purification step. In some such embodiments, the method produces a composition having at least 95% of one enantiomer and not more than 5% of the other enantiomer without employing a chiral purification step. In some such embodiments, the method produces a composition having at least 97% of one enantiomer and not more than 3% of the other enantiomer without employing a chiral purification step. In some such embodiments, the method produces a composition having at least 99% of one enantiomer and not more than 1% of the other enantiomer without employing a chiral purification step. In certain embodiments, a chiral purification step may be employed to removal residual enantiomeric impurities or to resolve a racemic product (for example, a diastereomerically enriched, or pure, product derived from a racemic starting material).

In certain embodiments, the present disclosure provides a method for forming a steroidal C9-C10 bond to yield a 9α, 13β anti-isomer, the method comprising the steps of (a) providing a compound of Formula (Y1) and (b) converting the compound of Formula (Y1) to a tetracyclic compound by a regio- and stereoselective cyclization reaction:

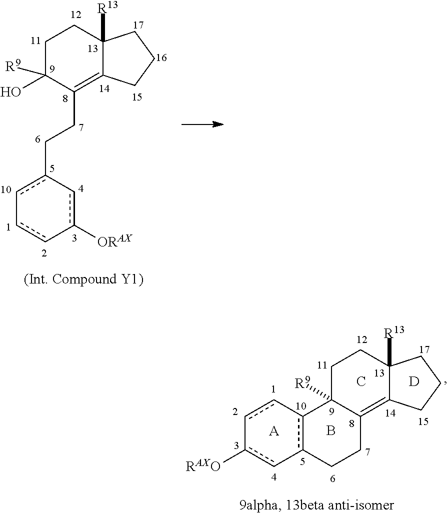

wherein R^{AX}, R⁹, and R¹³ are defined herein.

In certain embodiments, the present disclosure provides a method for forming a steroidal C9-C10 bond to yield a 9β, 13α anti-isomer, the method comprising the steps of (a) providing a compound of Formula (Y2) and (b) converting the compound of Formula (Y2) to a tetracyclic compound by a regio- and stereoselective cyclization reaction:

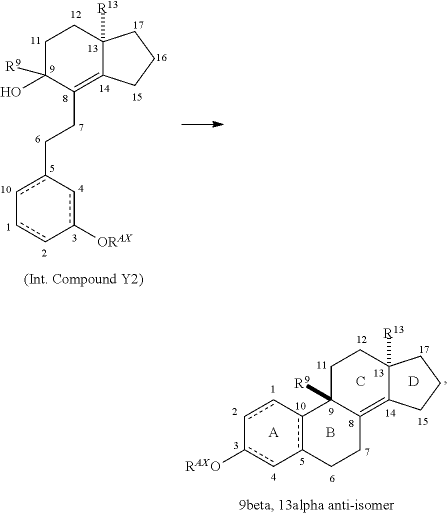

wherein R^{AX}, R¹⁰, R¹³, and R^{D} are defined herein.

The examples presented herein demonstrate the ability to generate a variety of tetracyclic terpenoid motifs through a regio- and stereoselective cyclization reaction. The cyclization technology generally produces tetracycles with C9/C13-anti stereochemistry. The tetracyclic products obtained from these reactions could be smoothly advanced to rearranged tetracyclic products bearing C10/C13 quaternary centers, demonstrating that the oxidative rearrangement is compatible with varying C9 substitution.

In one aspect, the present disclosure provides a method for shifting a substituent of a steroidal tetracycle from C9 to C10. The following general schemes are representative of a particular embodiment of the method:

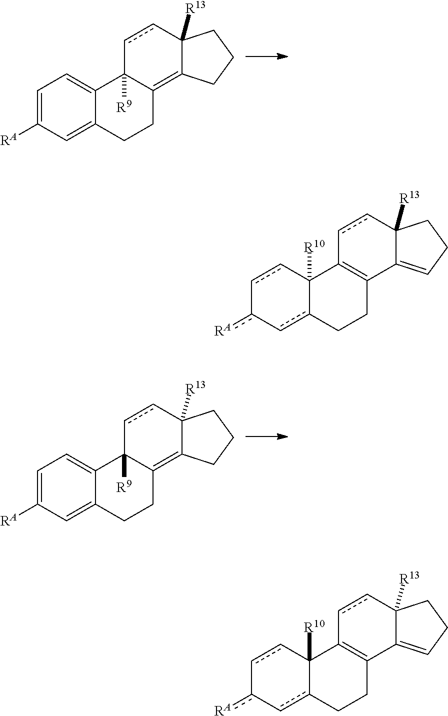

In certain embodiments, an oxidative rearrangement marked by a 1,2-alkyl shift from C9 to C10 is employed. In some such embodiments, concomitant establishment of an A-ring dienone is achieved.

In certain embodiments, the method is carried out in the presence of an oxidant. In certain embodiments, the oxidant is an aryliodine(III) carboxylate, such as is phenyliodo(III)diacetate (PIDA) or (bis(trifluoroacetate)iodo)benzene (PIFA). In some such embodiments, the oxidant is phenyliodo(III)diacetate (PIDA).

In certain embodiments, the method further comprises establishing a quaternary center at C10. In some such embodiments, a reaction is performed to results in stereospecific migration of the C9 substituent to C10. For example, an oxidative dearomatization and group-selective Wagner-Meerwein rearrangement may be performed. An exemplary oxidative dearomatization and group-selective Wagner-Meerwein rearrangement is depicted below.

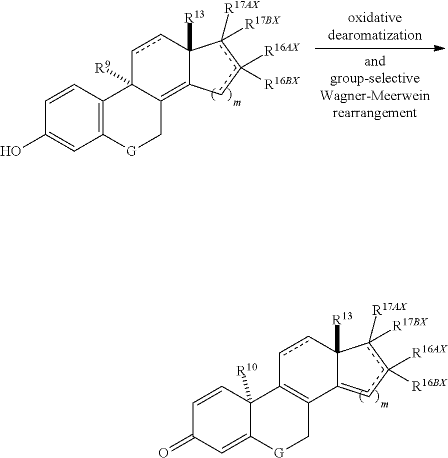

In one aspect, the present disclosure includes intermediate compounds useful in the preparation of, inter alia, steroidal tetracycles disclosed herein.

In certain embodiments, a substrate for the cyclization reaction comprises a steroidal C ring, a group that contains a nucleophile to participate in the cyclization process, and a tether connecting the steroidal C ring to the group that contains the nucleophile.

In certain embodiments, a substrate for the cyclization reaction comprises a tertiary allylic leaving group such as a halogen, an alcohol, an acetoxy, or a mesylate.

In one particular aspect, the present disclosure provides a substrate for the cyclization reaction that has a structure corresponding to Formula (Ei), Formula (Fi), Formula (Gi), Formula (Hi), Formula (Ji), Formula (Ki), Formula (Li), and Formula (Mi):

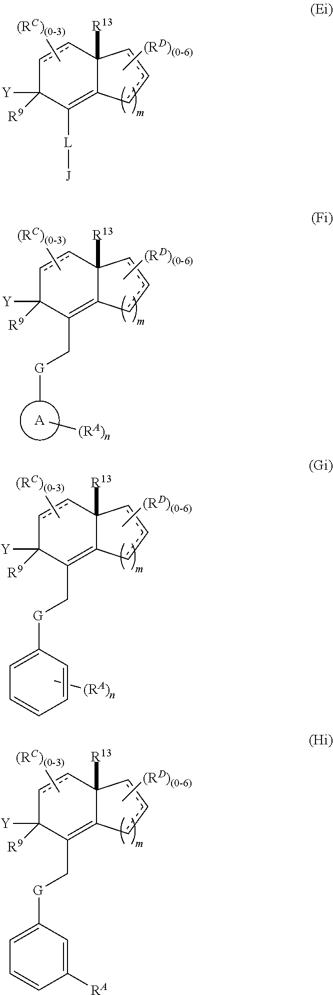

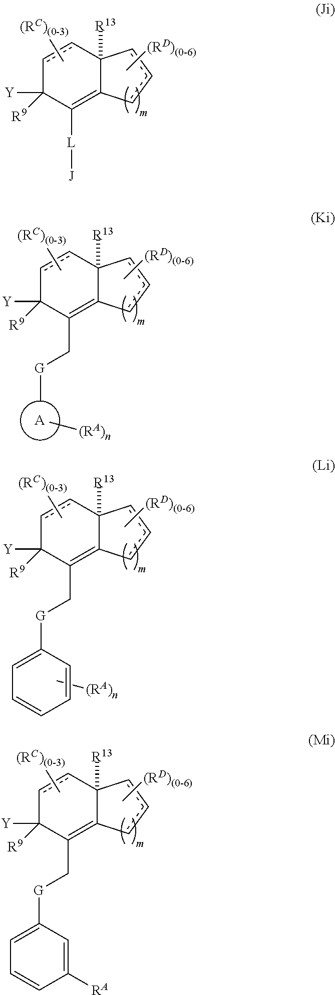

m is an integer selected from the group consisting of 1, 2, and 3;
Y is a leaving group such as a halogen, an alcohol, an acetoxy, or a mesylate;
L is a tether comprising 1, 2, or 3 chain atoms selected from the group consisting of carbon, nitrogen, oxygen, and sulfur;
J a group that contains a nucleophile to participate in the cyclization process;
G is a substituted or unsubstituted carbon atom, a substituted or unsubstituted nitrogen atom, an oxygen atom, or a sulfur atom;
Ring A represents a saturated or unsaturated carbocyclic or heterocyclic ring containing 5-7 ring atoms;
n is an integer selected from the group consisting of 0, 1, 2, 3, 4, 5, 6, 7, and 8;
each R^{A} is independently selected from the group consisting of hydrogen, C₁₋₁₀-alkyl, C₂₋₁₀-alkenyl, C₂₋₁₀-alkynyl, C₁₋₁₀-haloalkyl, halogen, oxo, —OR^{AX}, —SR^{AY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl,
   wherein R^{AX} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, —C(O)—O—C₁₋₁₀-alkyl, —C(O)—O—C₆₋₁₀-aryl, —C(O)—O-heteroaryl, —C(O)—NR^{Z1}R^{Z2}, —S(O)₂NR^{Z1}R^{Z2}, —S(O)₂R^{Z1}, C₆₋₁₀-aryl, or 5- to 10-membered heteroaryl,
   wherein R^{AY} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, C₆₋₁₀-aryl, or 5- to 10-membered heteroaryl,
   wherein each of R^{Z1} and R^{Z2} are independently hydrogen, C₁₋₆-alkyl, C₂₋₁₀-alkenyl, C₂₋₁₀-alkynyl, C₁₋₁₀-haloalkyl, —(CH₂)ₚ—C₆₋₁₀-aryl, —(CH₂)ₚ-5- to 10-membered heteroaryl, hydroxy, or C₁₋₆-alkoxy,
   wherein p is an integer selected from the group consisting of 0, 1, 2, and 3;
R⁹ and R¹³ are independently A—X^{A}—R^{X},
   wherein A is a C₁-C₁₄-alkylene, C₁-C₁₄-haloalkylene, C₂-C₁₄-alkenylene, C₂-C₁₄-haloalkenylene, C₂-C₁₄-alkynylene, C₂-C₁₄-haloalkynylene, each of which is optionally interrupted by one or more of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, or 5- to 10-membered heteroaryl;
   X^{A} is absent or selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, and 5- to 10-membered heteroaryl;
   R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)-heteroaryl, —C(O)-NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl;
   wherein R^{Z} is hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, C₆₋₁₀-aryl, or 5- to 10-membered heteroaryl and y is 0, 1, or 2;
R^{C} is hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, oxo, or X^{C}—R^{CC}, wherein X^{C} is selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, and R^{DD} is selected from the group consisting of hydrogen, C₁₋₆-alkyl, C₁₋₆-haloalkyl, C₂₋₁₀-alkenyl, C₂₋₆-haloalkenyl, C₂₋₁₀-alkynyl, C₂₋₆-haloalkynyl, C₁₋₁₀-hydroxyalkyl, —(CH₂)ₚ—C₆₋₁₀-aryl, and —(CH₂)ₚ-5- to 10-membered heteroaryl; or R^{C} is —OR^{CX} wherein R^{CX} is an oxygen protecting group such as methyl, tert-butyloxycarbonyl (BOC), methoxymethyl (MOM), tert-butyldimethylsilyl (TBS), tert-butyldiphenylsilyl (TBDPS), or tribenzylsilyl;
R^{D} is hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, oxo, or X^{D}—R^{DD}, wherein X^{D} is selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, and R^{DD} is selected from the group consisting of hydrogen, C₁₋₆-alkyl, C₁₋₆-haloalkyl, C₂₋₁₀-alkenyl, C₂₋₆-haloalkenyl, C₂₋₁₀-alkynyl, C₂₋₆-haloalkynyl, C₁₋₁₀-hydroxyalkyl, —(CH₂)ₚ—C₆₋₁₀-aryl, and —(CH₂)ₚ-5- to 10-membered heteroaryl; or R^{D} is —OR^{DX} wherein R^{DX} is an oxygen protecting group such as methyl, tert-butyloxycarbonyl (BOC), methoxymethyl (MOM), tert-butyldimethylsilyl (TBS), tert-butyldiphenylsilyl (TBDPS), or tribenzylsilyl;
wherein each [symbol] independently represents a single bond or a double bond;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₆-alkoxy.

In one particular aspect, the present disclosure provides a substrate for the cyclization reaction that has a structure corresponding to Formula (Eii), Formula (Fii), Formula (Gii), Formula (Hii), Formula (Jii), Formula (Kii), Formula (Lii), and Formula (Mii):

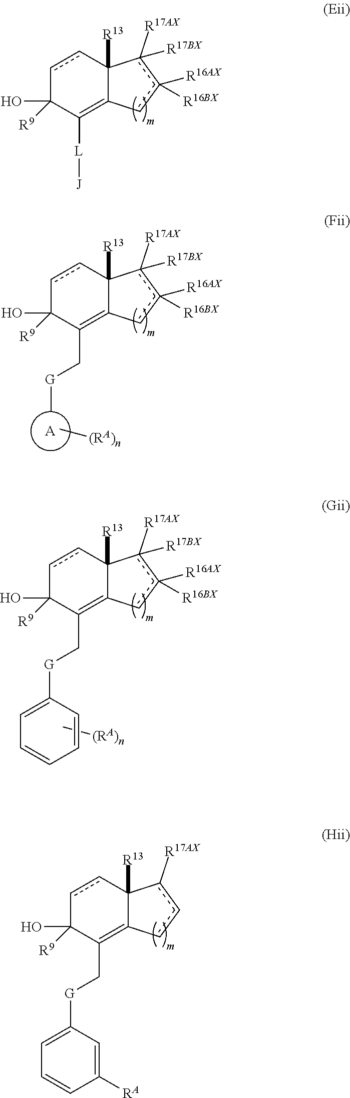

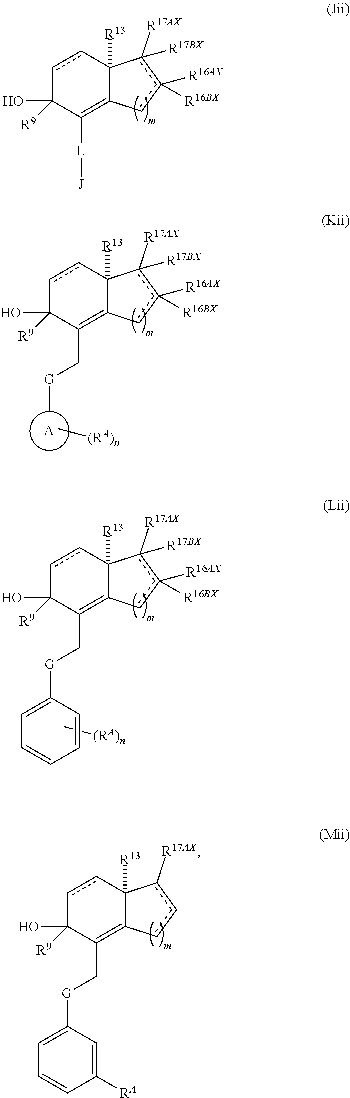

wherein
R^{16AX} is R^{16A} or —OR^{DX} and R^{16BX} is R^{16B} or —OR^{DX},
   wherein each of R^{16A} and R^{16B} are independently selected from the group consisting of hydrogen, oxo, and X^{D}—R^{DD};
R^{17AX} is R^{17A} or —OR^{DX} and R^{17BX} is R^{17B} or —OR^{DX},
   wherein each of R17A and R17B are independently selected from the group consisting of hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, C₁₋₁₀-alkyl-C(O), —C(O)—C₁₋₁₀-alkyl, —C(O)—C₁₋₁₀-hydroxyalkyl, —C(O)—C₁₋₁₀-alkyl-C₆₋₁₀-aryl, —C(O)—C₁₋₁₀-alkyl-heteroaryl, —C(O)—C₆₋₁₀-aryl, —C(O)-heteroaryl, —O—C(O)—C₁₋₆-alkyl, C₆₋₁₀-aryl, and 5- to 10-membered heteroaryl, or R^{17A} and R^{17B} together form an oxo;
wherein each [symbol] independently represents a single bond or a double bond;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₆-alkoxy.

In these cyclization substrates, the carbon atom bearing the R⁹ and leaving group (e.g., —OH) substituents is the C9 carbon atom and J, the A ring, and/or phenyl ring bearing the R^{A} substituent contains the C10 carbon atom. The cyclization reaction forms a C9-C10 bond and sets a quaternary center at C9.

In certain embodiments, the cyclization substrate is prepared according to known methods. For example, a cyclization substrate may be prepared from organometallic addition reaction to the corresponding enone. Structures containing such enones can be prepared through simple Robinson annulation technology and subsequent functionalization to alter substitution and/or level of unsaturation about the system.

In certain embodiments, the cyclization substrate is formed by reacting a suitable substituted cyclic enone with an organometallic reagent. In some such embodiments, the organometallic reagent is a Grignard reagent. In some such embodiments, the organometallic reagent is R⁹MgX, where X is a halogen.

In certain embodiments, the substituted cyclic enone is prepared by alkylation of a Hajos-Parrish ketone, a related carbocyclic system, or a derivative thereof. In some such embodiments, the alkylation reaction comprises:
(i) reacting a compound of Formula (i-a) with a compound of Formula (i-a′) to form the unsaturated alkylated hydrindane of Formula (i-b):

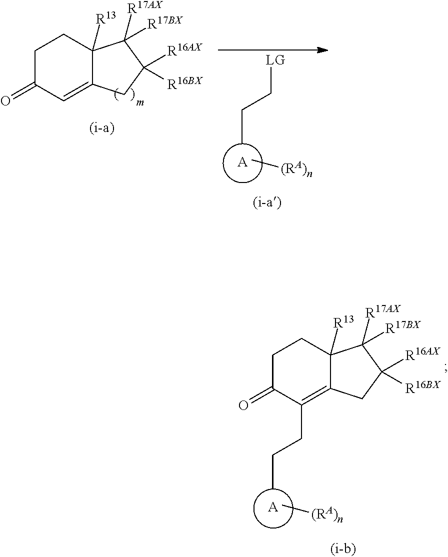

(ii) reacting a compound of Formula (ii-a) with a compound of Formula (ii-a′) to form the unsaturated alkylated hydrindane of Formula (ii-b):

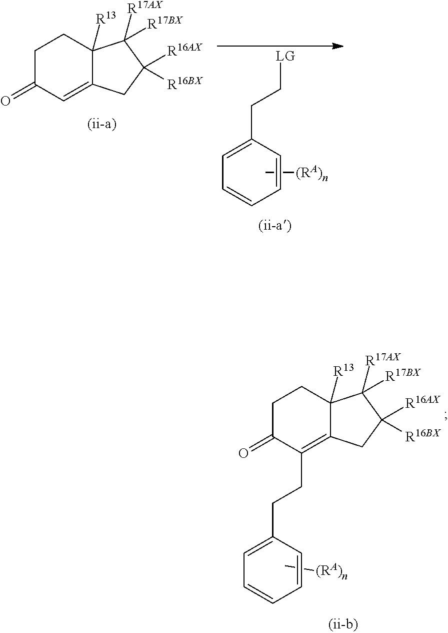

or
(iii) reacting a compound of Formula (iii-a) with a compound of Formula (iii-a′) to form the unsaturated alkylated hydrindane of Formula (iii-b):

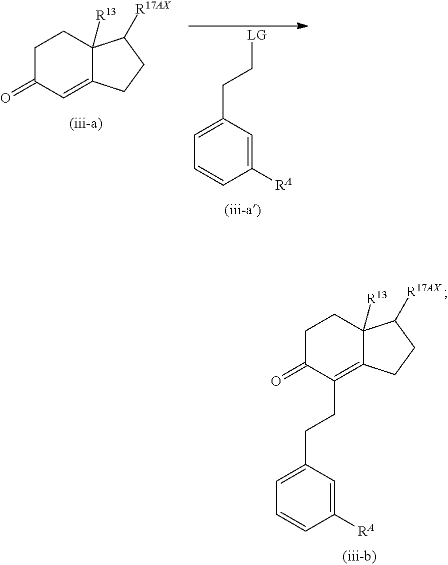

wherein LG is a leaving group such as halogen; —O—Ar¹, where Ar¹ is a substituted or unsubstituted C₆₋₁₀-aryl or 5- to 10-membered heteroaryl, in particular —O-phenyl; or —OSO₂R^{1a}, wherein R^{1a} is aryl, such as p-tolyl or phenyl, alkyl such as methyl or ethyl, fluoroalkyl such as trifluoromethyl, perfluorobutyl (C₄F₉), perfluoropentyl, perfluorohexyl, and perfluorooctyl, -fluoroalkyl-O-fluoroalkyl such as perfluoroethoxyethyl, —N(alkyl)₂, fluoro, or imidazolyl. In one particular embodiment, LG is p-toluenesulfonate (OTs).

In one particular embodiment, at least one of R^{17AX} or R^{17BX} is —OR^{DX} and R^{DX} is an oxygen protecting group, preferably tert-butyldimethylsilyl (TBS), or a saturated or unsaturated, optionally substituted, C₁₋₆-alkyl or C₆₋₁₀-aryl group.

### C. EXEMPLARY COMPOUNDS

In one aspect, this disclosure provides a compound or salt or prodrug thereof, wherein the compound either has a structure corresponding to Formula (I-A) or Formula (II-A), or could be transformed to such structures by methods well known to those skilled in the art of synthetic organic chemistry:

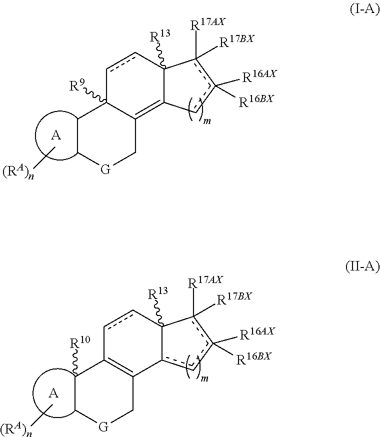

In some such embodiments, the compound either has a structure corresponding to Formula (I-B) or Formula (II-B), or could be transformed to such structures by methods well known to those skilled in the art of synthetic organic chemistry:

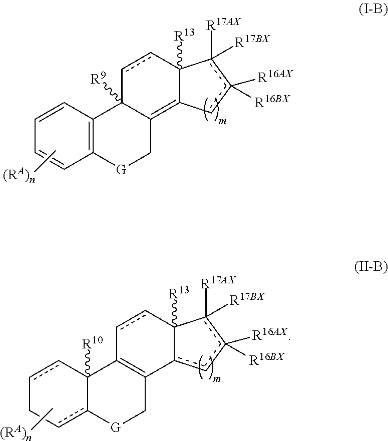

In some such embodiments, the compound either has a structure corresponding to Formula (I-C) or Formula (II-C), or could be transformed to such structures by methods well known to those skilled in the art of synthetic organic chemistry:

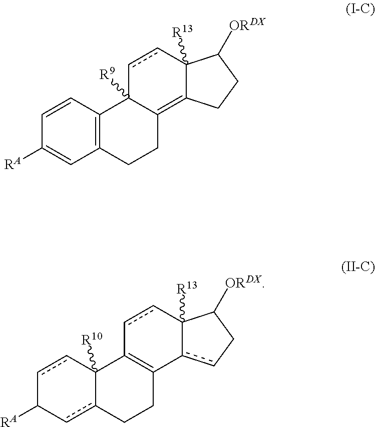

In certain embodiments, the substituents of Formula (I-A), Formula (I-B), or Formula (I-C) attached to carbon C9 and carbon C13 by [symbol] have different orientations (e.g., one [symbol] and the other [symbol] ).

In certain embodiments, the substituents of Formula (II-A), Formula (II-B), or Formula (II-C) attached to carbon C10 and carbon C13 by [symbol] have different orientations (e.g., one [symbol] and the other [symbol] ).

### D. METHODS OF USE

In at least one aspect, the present disclosure includes a method for treating or preventing a disease that is treatable or preventable by modulation of a nuclear hormone receptor in a subject in need of such treatment or prevention. Exemplary nuclear hormone receptors include estrogen receptor beta (ERβ), androgen receptor (AR), and glucocorticoid receptor (GR). Exemplary diseases include proliferative diseases include cancers (i.e., “malignant neoplasms”), benign neoplasms, angiogenesis, inflammatory diseases, and autoimmune diseases. In particular, exemplary cancers that may be treated or prevented include breast cancer, prostate cancer, ovarian cancer, acute myeloid leukemia, and glioma.

The method comprises administering to a patient in need thereof a therapeutically effective amount of a compound described herein or a pharmaceutically acceptable salt or prodrug thereof. In some embodiments, the compound (or pharmaceutically acceptable salt thereof) is administered orally.

In certain embodiments, for any of the aforementioned aspects, the subject is a mammal. In some such embodiments, the mammal is a human.

The preferred total daily dose of the compound or salt (administered in single or divided doses) is typically from about 0.001 to about 100 mg/kg, more preferably from about 0.001 to about 30 mg/kg, and even more preferably from about 0.01 to about 10 mg/kg (i.e., mg of the compound or salt per kg body weight). In certain embodiments, dosage unit compositions contain such amounts or submultiples thereof to make up the daily dose. In many instances, the administration of the compound or salt will be repeated a plurality of times. In certain embodiments, multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular compound or salt used; whether a drug delivery system is utilized; and whether the compound or salt is administered as part of a drug combination. Thus, the dosage regimen actually employed can vary widely, and therefore, can derive from the preferred dosage regimen set forth above.

### E. COMPOSITIONS

In at least one aspect, the present disclosure includes compositions comprising a compound described herein or a pharmaceutically acceptable salt or prodrug thereof. In certain embodiments, the composition comprises one or more conventional pharmaceutically acceptable excipients.

In at least one aspect, the present disclosure includes compositions comprising an enantiomeric compound described herein. In certain embodiments, the composition is enantiomerically pure or enriched. For example, the composition may comprise at least 85% of one enantiomer and not more than 15% of the other enantiomer; alternatively, at least 90% of one enantiomer and not more than 10% of the other enantiomer; alternatively, at least 95% of one enantiomer and not more than 5% of the other enantiomer; alternatively, at least 97% of one enantiomer and not more than 3% of the other enantiomer; or alternatively, at least 99% of one enantiomer and not more than 1% of the other enantiomer. In certain embodiments, the composition is substantially free of enantiomeric impurities. In some such embodiments, the composition is free of any detectable amount of an enantiomeric impurity.

Pharmaceutical compositions disclosed herein comprise a compound disclosed herein or a pharmaceutically acceptable salt or prodrug thereof. In some embodiments, the pharmaceutical composition is an oral dosage form, preferably a solid oral dosage form (e.g., a tablet). In some such embodiments, the solid oral dosage form may comprise pharmaceutically acceptable excipients such as excipients that function as binders, glidants, lubricants, and fillers. Thus, a solid oral dosage form comprising a compound disclosed herein or a pharmaceutically acceptable salt thereof further optionally comprises one or more conventional pharmaceutically acceptable excipients.

It will be readily apparent to those skilled in the art that other suitable modifications and adaptations of the compositions and methods of the invention described herein may be made using suitable equivalents without departing from the scope of the invention or the embodiments disclosed herein.

The compounds, compositions, and methods described herein will be better understood by reference to the following examples, which are included as an illustration of and not a limitation upon the scope of the invention.

### F. EXAMPLES

### Materials and Methods

All reactions were conducted in flame-dried glassware under a nitrogen atmosphere with dry solvents, unless otherwise noted. All reagents and starting materials were purchased from commercial sources and used as supplied, unless otherwise indicated.

### Example 1

Existing technology is capable of forging a C9-C10 bond in a steroidal skeleton and providing a substituent at C9 that is derived from TMS-alkynes. This Example is directed to an alternate scheme that has certain advantages over existing technology. In particular, the steroidal skeleton is forged in a manner that allows for facile incorporation of molecular substituents at C17, and the substituent at C9 ultimately derived from a simple organometallic reagent (for example, a Grignard reagent). The ability to access steroidal systems that contain both varied substitution at C9 and functionality at C17 is very significant.

### Synthesis of Cyclization Substrate

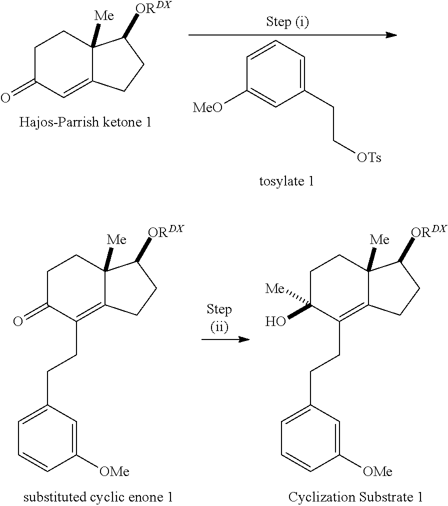

Step (i) was an alkylation reaction to convert a Hajos-Parrish ketone, where R^{DX} is a protecting group such as tert-butyl(dimethyl)silyl (TBS), to suitably substituted cyclic enone 1.

To a stirring solution of TBS-protected Hajos-Parrish ketone 1 (508.7 mg, 1.8 mmol, 1.0 equiv) in DME (1.8 mL) was added NaH (60% dispersion in mineral oil, 108.9 mg, 2.7 mmol, 1.5 equiv) at rt. The reaction mixture was heated to 65° C. and stirred for 20 h. After the indicated time, tosylate 2 (611.2 mg, 2.0 mmol, 1.1 equiv) was dissolved in DME (1.8 mL) and the resulting solution was added dropwise to the reaction mixture over 15 min at 65° C. The resulting solution was stirred for 1 h at 65° C., during which time tosylate 1 was consumed (as indicated by TLC analysis). The reaction was then cooled to 0° C. and quenched by the addition of a saturated aqueous solution of NaH₂PO₄ (2 mL). The reaction mixture was warmed to room temperature before the two phases were separated, and the aqueous phase was extracted with DCM. The combined organic phase was washed with brine and dried over Na₂SO₄, filtered, and concentrated in vacuo. The crude concentrate was purified by flash chromatography with a Biotage® Sfar Silica HC D 50 g column and a gradient from 0-12% EtOAc in hexanes with an additive of 5% DCM to afford substituted cyclic enone 1 (388.7 mg, 0.9 mmol, 52%) as a yellow oil [55% yield based on recovered starting material; recovered 28.7 mg (0.1 mmol) Hajos-Parrish ketone 1].

Data for substituted cyclic enone 1: R_{f}=0.32 (10% EtOAc in hexanes); ¹H NMR (600 MHZ, CDCl₃) δ 7.16 (t, J=7.8 Hz, 1H), 6.72 (t, J=6.6 Hz, 2H), 6.66 (s, 1H), 3.78 (s, 3H), 3.59 (t, J=9.3, 1H), 2.62 (t, J=7.4 Hz, 2H), 2.59-2.51 (m, 1H), 2.45-2.36 (m, 3H), 2.14 (dd, J=19.6, 11.1 Hz, 1H), 2.06-1.94 (m, 2H), 1.81-1.74 (m, 1H), 1.70-1.60 (m, 2H), 1.01 (s, 3H), 0.89 (s, 9H), 0.06 (d, J=3.8 Hz, 6H); ¹³C NMR (150 MHz, CDCl₃): δ 198.62, 169.38, 159.57, 143.89, 132.13, 129.18, 121.49, 114.81, 111.16, 80.99, 55.30, 45.65, 34.76, 34.33, 33.79, 29.93, 27.92, 25.89, 25.24, 18.15, 15.48, −4.34, −4.78; IR (neat, cm⁻¹): 2957 (s), 2925 (s), 2860 (m), 1657 (s), 1603 (w), 1585 (w), 1495 (w), 1464 (w), 1349 (w), 1249 (m), 1155 (w), 1114 (s), 1051 (w), 1027 (w), 903 (w), 868 (w), 837 (m), 781 (m); HRMS (ESI-TOF) (m/z): [M+H]⁺ calcd for C₂₅H₃₉O₃Si 415.2668; found, 415.2661; [α]₅₈₉^{22.1}: +18.4463 (c=0.85, CHCl₃).

In step (ii), cyclic enone 1 was reacted with an organometallic reagent, R⁹MgX, where R⁹ is Me and X is a halogen.

To a stirring solution of cyclic enone 1 (53.1 mg, 0.12 mmol, 1.0 equiv) in THF (1.2 mL) was added MeMgBr (53.6 μL, 0.17 mmol, 1.3 equiv, 3.1 M in diethyl ether) dropwise at −30° C. The reaction mixture was stirred at −30° C. for 2 h and then more MeMgBr (30.0 μL, 0.09 mmol, 0.7 equiv, 3.1 M in diethyl ether) was added dropwise. The reaction mixture was stirred for 1 h at −30° C. before warming to room temperature. After stirring at room temperature for 21 h, the mixture was cooled to 0° C. and then the reaction was quenched by the slow addition of a saturated aqueous solution of NH₄Cl (0.2 mL). The reaction mixture was warmed to room temperature before water (0.5 mL) was added. The two phases were separated, and the aqueous phase was extracted with EtOAc. The combined organic phase was washed with brine and dried over Na₂SO₄, filtered, and concentrated in vacuo. The crude concentrate was purified by flash chromatography with a Biotage® Sfar Silica HC D 10 g column. The column was deactivated with 50 mL of a 5% triethylamine in hexanes (by volume) solution before use and then purification was done with a gradient from 5-20% EtOAc in hexanes to afford cyclization substrate 1 (38.7 mg, 0.09 mg, 70%) as a colorless oil [91% yield based on recovered starting material; recovered 12.3 mg (0.03 mmol) cyclic enone 1].

Data for cyclization substrate 1: R^{f}=0.28 (15% EtOAc in hexanes); ¹H NMR (600 MHZ, CDCl₃): δ 7.20 (t, J=8.1 Hz, 1H), 6.79 (d, J=7.5 Hz, 1H), 6.74 (d, J=6.3 Hz, 2H), 3.80 (s, 3H), 3.52 (t, J=8.9 Hz, 1H), 2.75-2.61 (m, 2H), 2.42-2.24 (m, 3H), 2.16 (dt, J=17.2, 8.5 Hz, 1H), 1.92-1.65 (m, 5H), 1.32 (t, J=13.4, 1H), 1.22 (s, 3H), 0.99 (s, 3H), 0.89 (s, 9H), 0.03 (s, 6H); ¹³C NMR (150 MHz, CDCl₃): δ 159.76, 144.46, 142.22, 134.37, 129.42, 121.18, 114.60, 111.26, 81.59, 73.80, 55.31, 45.04, 37.61, 36.32, 34.21, 30.52, 29.76, 27.72, 25.96, 24.07, 18.21, 17.44, −4.24, −4.64; IR (neat, cm⁻¹): 3451 (br), 2957 (s), 2933 (s), 2846 (m), 1609 (w), 1456 (m), 1366 (w), 1265 (m), 1119 (s), 1029 (w), 876 (w), 838 (s), 786 (m); HRMS (ESI-TOF) (m/z): [M−OH]⁺ calcd for C₂₆H₄₁O₂Si 413.2876; found, 413.2867; [α]₅₈₉^{22.2}: +14.0087 (c=0.23, CHCl₃).

Synthesis of 9α-substituted Compound:

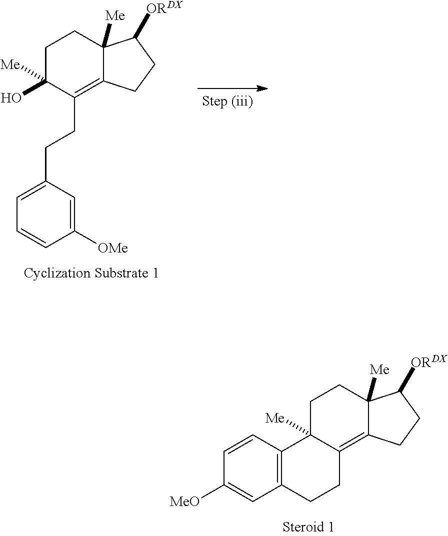

Step (iii) was an acid mediated regio- and stereoselective cyclization reaction. The acid was SnCl₄. Steroid 1 was obtained with high selectivity (rs≥20:1; ds≥20:1).

To a stirring solution of cyclization substrate 1 (80.8 mg, 0.2 mmol, 1.0 equiv) in DCM (1.2 mL) at −78° C. was added SnCl₄ (0.6 mL, 0.6 mmol, 3.0 equiv, 1.0 M in DCM) dropwise. The resulting mixture was stirred at −78° C. for 1 h, then warmed to −30° C. The reaction was quenched by the addition of a saturated aqueous solution of NaHCO₃ (1 mL). The resulting mixture was warmed to room temperature before the two phases were separated, and the aqueous phase was extracted with DCM. The combined organic phase was washed with water and brine then dried over Na₂SO₄, filtered, and concentrated in vacuo. The crude concentrate was purified by flash chromatography with a Biotage® Sfar Silica HC D 10 g column and a gradient from 0-10% EtOAc in hexanes to afford steroid 1 (55.3 mg, 0.1 mmol, 72%) as a yellow oil. No evidence was found for the presence of another diastereomer in the ¹H NMR of the crude material (ds≥20:1).

Data for steroid 1: R^{f}=0.62 (10% EtOAc in hexanes); ¹H NMR (600 MHZ, CDCl₃): δ 7.21 (d, J=8.6 Hz, 1H), 6.76 (d, J=8.3 Hz, 1H), 6.57 (s, 1H), 3.77 (s, 3H), 3.62 (t, J=8.7 Hz, 1H), 2.85 (ddd, J=16.1, 5.9, 2.6 Hz, 1H), 2.71 (ddd, J=16.4, 12.3, 5.7 Hz, 1H), 2.46 (ddd, J=13.1, 5.8, 2.6 Hz, 1H), 2.38-2.20 (m, 3H), 2.09 (dt, J=13.2, 3.5 Hz, 1H), 1.94-1.87 (m, 1H), 1.83 (t, J=13.9 Hz, 1H), 1.74-1.64 (m, 2H), 1.47 (t, J=13.9 Hz, 1H), 1.29 (s, 3H), 0.91 (s, 9H), 0.84 (s, 3H), 0.06 (d, J=7.8, 6H); ¹³C NMR (150 MHz, CDCl₃): δ 157.08, 140.30, 137.39, 135.98, 132.90, 127.37, 113.07, 112.50, 82.07, 55.29, 44.20, 38.35, 34.73, 32.26, 32.06, 31.39, 30.64, 26.00, 24.14, 23.79, 18.24, 17.67, −4.22, −4.58; IR (neat, cm⁻¹): 2964 (s), 2925 (s), 2857 (s), 1609 (m), 1492 (m), 1457 (m), 1377 (w), 1249 (s), 1103 (s), 1034 (m), 909 (w), 872 (m), 839 (m), 809 (w), 774 (m), 667 (w); HRMS (ESI-TOF) (m/z): [M+H]⁺ calcd for C₂₆H₄₁O₂Si 413.2876; found, 413.2861; [α]₅₈₉^{21.9}: +98.4949 (c=0.295, CHCl₃).

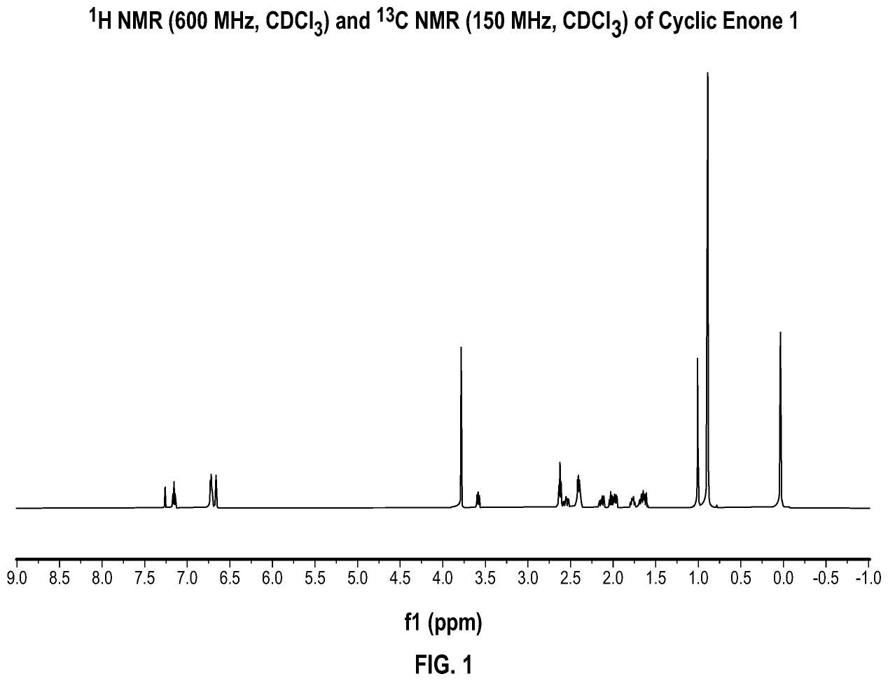

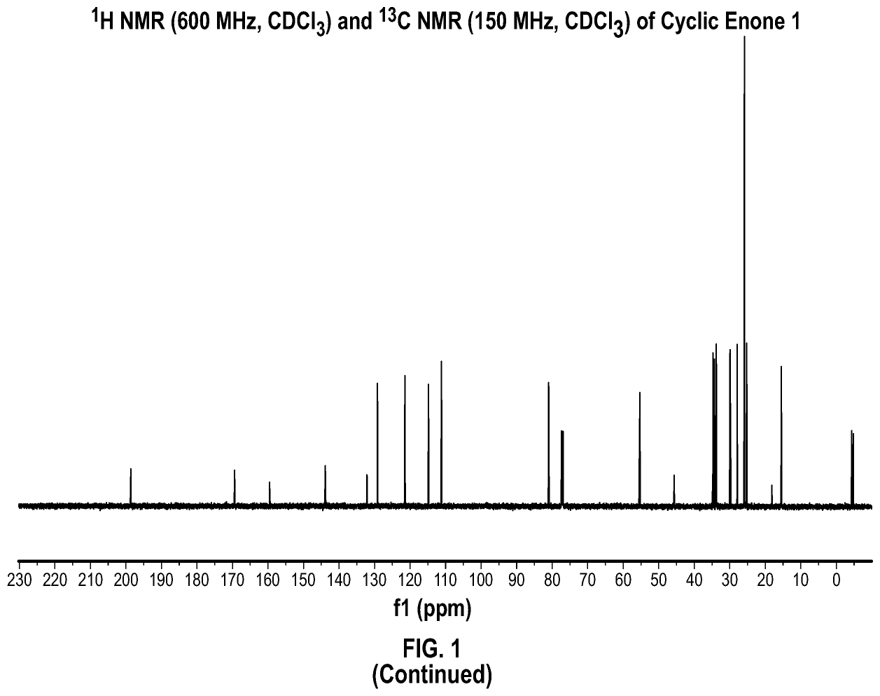

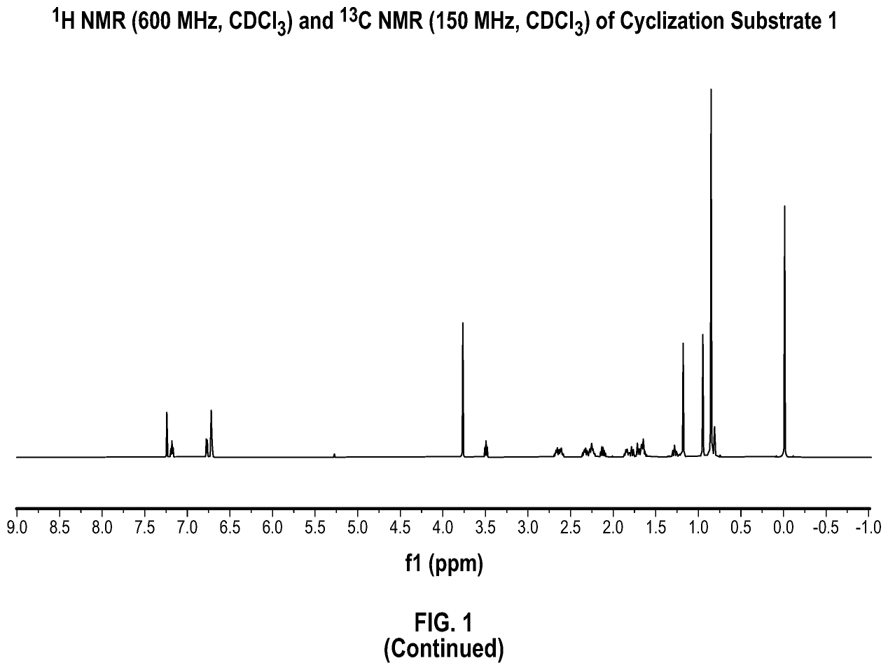

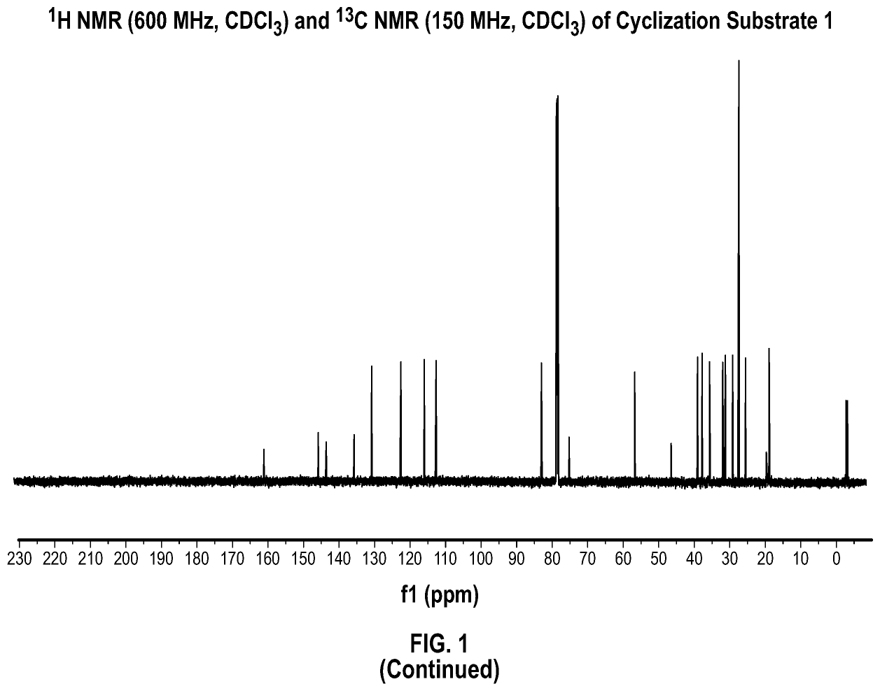

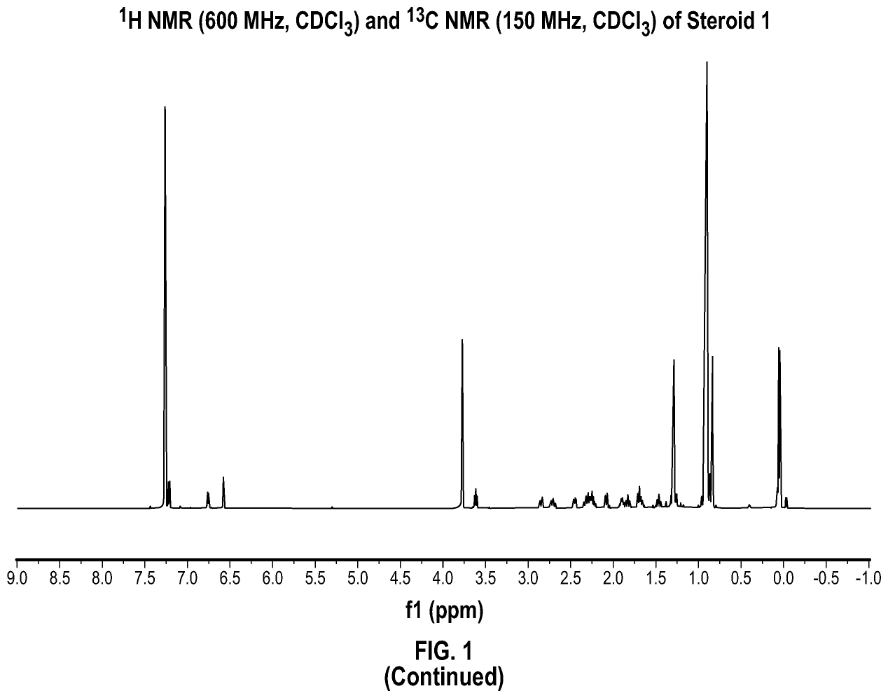

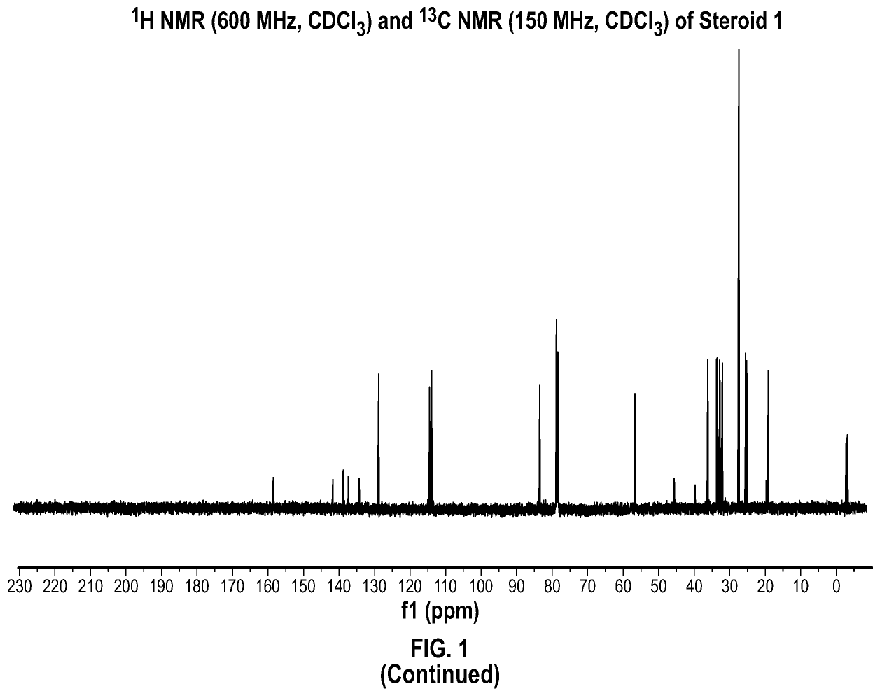

Compounds were characterized by nuclear magnetic resonance (NMR) spectroscopy. ¹H NMR and ¹³C NMR spectra for cyclic enone 1, cyclization substrate 1, and steroid 1 are shown in FIG. 1.

¹H NMR data was recorded on a Bruker Avance III 500 MHz NMR spectrometer (TBI probe) and/or a Bruker Avance III 600 MHz spectrometer (BBFO probe). ¹H chemical shifts are reported in parts per million (ppm, δ scale) downfield from tetramethylsilane and are referenced to the residual protium in CDCl₃ (7.26 ppm). NMR coupling constants are measured in Hertz (Hz), and splitting patterns are indicated as follows: s, singlet; d, doublet; dd, doublet of doublets; ddd, doublet of doublets of doublets; dt, doublet of triplets; t, triplet; m, multiplet. ¹³C {¹H decoupled} NMR data were recorded at 150 MHz on a Bruker Avance III 600 MHz spectrometer (BBFO probe). ¹³C NMR chemical shifts are reported in parts per million (ppm, δ scale) and are referenced to the central line of the carbon resonance of the solvent CDCl₃ (77.16) ppm. Structural assignments for new compounds were supported by two-dimensional NMR experiments (COSY, HSQC, HMBC, and NOESY) recorded on a Bruker Avance III 600 MHz spectrometer (BBFO probe) as well as one-dimensional NOE experiments recorded on a Bruker Avance III 500 MHz NMR spectrometer (TBI probe).

In this Example, the substituent at C9 was derived from a simple organometallic and the method allowed for functionality to be installed at C17.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A method for preparing a C9-alpha-substituted or a C9-beta-substituted steroid-like compound (steroid numbering), the method comprising the steps of providing a tertiary allylic substrate and performing a regio- and stereoselective cyclization reaction to form a C9—C10 bond and to set a quaternary center at C9, wherein the reaction is understood to likely proceed through a cationic intermediate to yield the C9-alpha-substituted steroid-like compound or the C9-beta-substituted steroid-like compound.

2. The method of claim 1, wherein the cyclization reaction is mediated by a protic or Lewis acid, such as SnCl₄.

3. The method of claim 1, wherein the substrate for the cyclization reaction comprises a steroidal C ring, a group that contains a nucleophile to participate in the cyclization process, and a tether connecting the steroidal C ring to the group that contains the nucleophile.

4. The method of claim 3, wherein the cyclization substrate comprises a tertiary allylic leaving group such as a halogen, an alcohol, an acetoxy, or a mesylate.

5. The method of claim 1, wherein a substrate for the cyclization reaction has a structure corresponding to Formula (Ei), Formula (Fi), Formula (Gi), Formula (Hi), Formula (Ji), Formula (Ki), Formula (Li), and Formula (Mi): 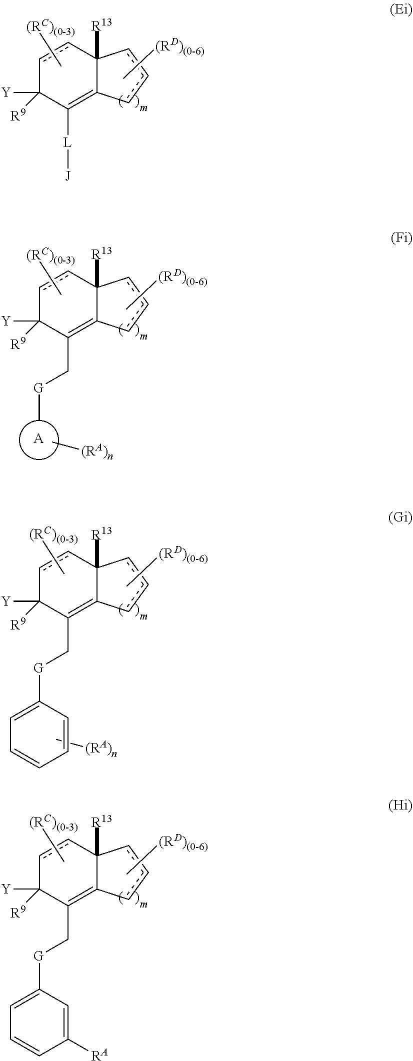 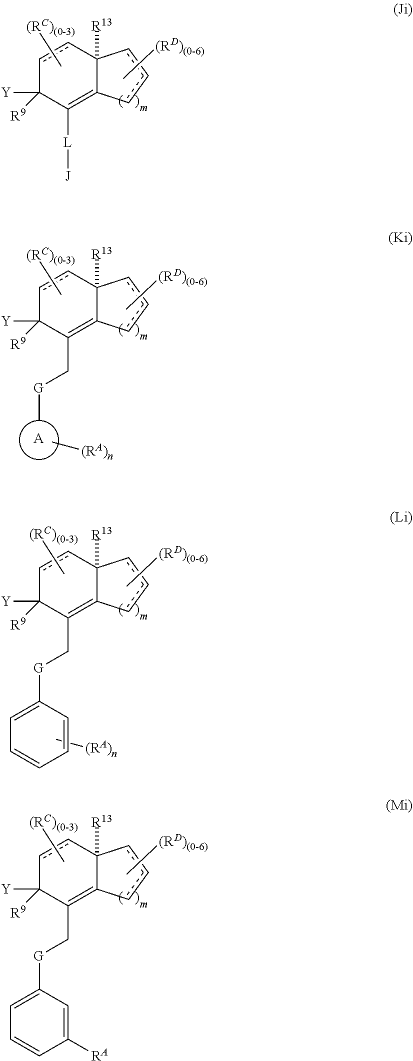
m is an integer selected from the group consisting of 1, 2, and 3;
Y is a leaving group such as a halogen, an alcohol, an acetoxy, or a mesylate;
L is a tether comprising 1, 2, or 3 chain atoms selected from the group consisting of carbon, nitrogen, oxygen, and sulfur;
J a group that contains a nucleophile to participate in the cyclization process;
G is a substituted or unsubstituted carbon atom, a substituted or unsubstituted nitrogen atom, an oxygen atom, or a sulfur atom;
Ring A represents a saturated or unsaturated carbocyclic or heterocyclic ring containing 5-7 ring atoms;
n is an integer selected from the group consisting of 0, 1, 2, 3, 4, 5, 6, 7, and 8;
each R^{A} is independently selected from the group consisting of hydrogen, C₁₋₁₀-alkyl, C₂₋₁₀-alkenyl, C₂₋₁₀-alkynyl, C₁₋₁₀-haloalkyl, halogen, oxo, —OR^{AX}, —SR^{AY}, —S(O)₂NR^{Z1}R^{Z2}, —S(O)₂R^{Z1}, —S(O)R^{Z1}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl,
wherein R^{AX} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, —C(O)—O—C₁₋₁₀-alkyl, —C(O)—O—C₆₋₁₀-aryl, —C(O)—O-heteroaryl, —C(O)—NR^{Z1}R^{Z2}, —S(O)₂NR^{Z1}R^{Z2}, —S(O)₂R^{Z1}, C₆₋₁₀-aryl, or 5- to 10-membered heteroaryl,
wherein R^{AY} is hydrogen, C₁₋₆-alkyl, C₂₋₁₀-alkenyl, C₂₋₁₀-alkynyl, C₁₋₁₀-haloalkyl, —C(O)—C₁₋₁₀-alkyl, —C(O)—C₆₋₁₀-aryl, —C(O)-heteroaryl, C₆₋₁₀-aryl, or 5- to 10-membered heteroaryl,
wherein each of R^{Z1} and R^{Z2} are independently hydrogen, C₁₋₆-alkyl, C₂₋₁₀-alkenyl, C₂₋₁₀-alkynyl, C₁₋₁₀-haloalkyl, —(CH₂)p-C₆₋₁₀-aryl, —(CH₂)p-5- to 10-membered heteroaryl, hydroxy, or C₁₋₆-alkoxy,
wherein p is an integer selected from the group consisting of 0, 1, 2, and 3;
R⁹ and R¹³ are independently A—X^{A}—R^{X},
wherein A is a C₁-C₁₄-alkylene, C₁-C₁₄-haloalkylene, C₂-C₁₄-alkenylene, C₂-C₁₄-haloalkenylene, C₂-C₁₄-alkynylene, C₂-C₁₄-haloalkynylene, each of which is optionally interrupted by one or more of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, or 5- to 10-membered heteroaryl;
X^{A} is absent or selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, C₆₋₁₀-aryl, and 5- to 10-membered heteroaryl;
R^{X} is selected from the group consisting of hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, —C(O)—C₁₋₆-alkyl, —C(O)—C₆₋₁₀-aryl, —C(O)-heteroaryl, —C(O)—NR^{Z1}R^{Z2}, —S(O)₂NR^{Z1}R^{Z2}, —NR^{Z1}R^{Z2}, —N(R^{Z1})C(O)R^{Z2}, —N(R^{Z1})S(O)₂R^{Z2}, C₆₋₁₀-aryl, and 5- to 10-membered heteroaryl;
wherein R^{Z} is hydrogen, C₁₋₆-alkyl, C₁₋₆-haloalkyl, C₂₋₆-alkenyl, C₂₋₆-haloalkenyl, C₂₋₆-alkynyl, C₂₋₆-haloalkynyl, C₃₋₇-cycloalkyl, C₆₋₁₀-aryl, or 5- to 10-membered heteroaryl and y is 0, 1, or 2;
R^{C} is hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, oxo, or X^{C}—R^{CC}, wherein X^{C} is selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, and R^{DD} is selected from the group consisting of hydrogen, C₁₋₆-alkyl, C₁₋₆-haloalkyl, C₂₋₁₀-alkenyl, C₂₋₆-haloalkenyl, C₂₋₁₀-alkynyl, C₂₋₆-haloalkynyl, C₁₋₁₀-hydroxyalkyl, —(CH₂)ₚ—C₆₋₁₀-aryl, and —(CH₂)ₚ-5- to 10-membered heteroaryl; or R^{C} is —OR^{CX} wherein R^{CX} is an oxygen protecting group such as methyl, tert-butyloxycarbonyl (BOC), methoxymethyl (MOM), tert-butyldimethylsilyl (TBS), tert-butyldiphenylsilyl (TBDPS), or tribenzylsilyl;
R^{D} is hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, oxo, or X^{D}—R^{DD}, wherein X^{D} is selected from the group consisting of —O—, —NR^{Z}—, —C(O)—, —C(O)O—, —OC(O)—, —C(O)NR^{Z}—, —NR^{Z}C(O)—, —S(O)_{y}—, —S(O)_{y}NR^{Z}—, —NR^{Z}S(O)_{y}—, —C(S)NR^{Z}—, —NR^{Z}C(S)—, and R^{DD} is selected from the group consisting of hydrogen, C₁₋₆-alkyl, C₁₋₆-haloalkyl, C₂₋₁₀-alkenyl, C₂₋₆-haloalkenyl, C₂₋₁₀-alkynyl, C₂₋₆-haloalkynyl, C₁₋₁₀-hydroxyalkyl, —(CH₂)ₚ—C₆₋₁₀-aryl, and —(CH₂)ₚ-5- to 10-membered heteroaryl; or R^{D} is —OR^{DX} wherein R^{DX} is an oxygen protecting group such as methyl, tert-butyloxycarbonyl (BOC), methoxymethyl (MOM), tert-butyldimethylsilyl (TBS), tert-butyldiphenylsilyl (TBDPS), or tribenzylsilyl;
wherein each [symbol] independently represents a single bond or a double bond;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₆-alkoxy.

6. The method of claim 4, wherein the cyclization substrate has a structure corresponding to Formula (Eii), Formula (Fii), Formula (Gii), Formula (Hii), Formula (Jii), Formula (Kii), Formula (Lii), and Formula (Mii): 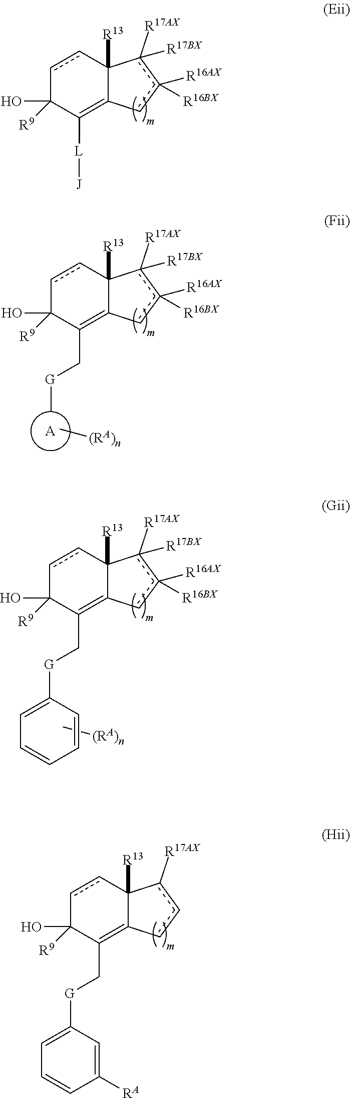 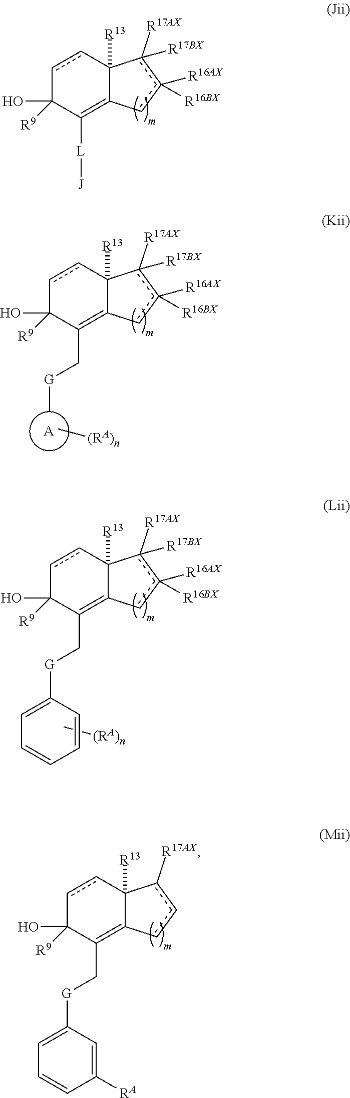
wherein
R^{16AX} is R^{16A} or —OR^{DX} and R^{16BX} is R^{16B} or —OR^{DX},
wherein each of R^{16A} and R^{16B} are independently selected from the group consisting of hydrogen, oxo, and X^{D}—R^{DD};
R^{17AX} is R^{17A} or —OR^{DX} and R^{17BX} is R^{17B} or —OR^{DX},
wherein each of R^{17A} and R^{17B} are independently selected from the group consisting of hydrogen, C₁₋₁₀-alkyl, C₂₋₁₀-alkenyl, C₂₋₁₀-alkynyl, C₁₋₁₀-haloalkyl, halogen, hydroxy, C₁₋₆-alkoxy, C₁₋₁₀-alkyl-C(O), —C(O)—C₁₋₁₀-alkyl, —C(O)—C₁₋₁₀-hydroxyalkyl, —C(O)—C₁₋₁₀-alkyl-C₆₋₁₀-aryl, —C(O)—C₁₋₁₀-alkyl-heteroaryl, —C(O)—C₆₋₁₀-aryl, —C(O)-heteroaryl, —O—C(O)—C₁₋₆-alkyl, C₆₋₁₀-aryl, and 5- to 10-membered heteroaryl, or R^{17A} and R^{17B} together form an oxo;
wherein each [symbol] independently represents a single bond or a double bond;
wherein any C₆₋₁₀-aryl or 5- to 10-membered heteroaryl is optionally substituted with one or more halogen, hydroxy, C₁₋₆-alkyl, C₁₋₆-haloalkyl, or C₁₋₆-alkoxy.

7. The method of claim 1, wherein the C9-alpha-substituted steroid-like compound or the C9-beta-substituted steroid-like compound is a tetracyclic compound.

8. The method of claim 1, further comprising an oxidative dearomatization and group-selective Wagner-Meerwein rearrangement that establishes a quaternary center at C10.

9. The method of claim 3, wherein the cyclization substrate is formed by reacting a suitable substituted cyclic enone with a nucleophile, such as an organometallic reagent, preferably a Grignard reagent, such as R⁹MgX, where X is a halogen.

10. The method of claim 9, wherein the enone substrate is prepared by alkylation of a Hajos-Parrish ketone, a related carbocyclic system, or a derivative thereof.

11. A method for preparing (a) a C9-alpha-substituted tetracyclic compound (steroid numbering), the method comprising a regio- and stereoselective cyclization reaction depicted in Scheme (1), Scheme (2), or Scheme (3): 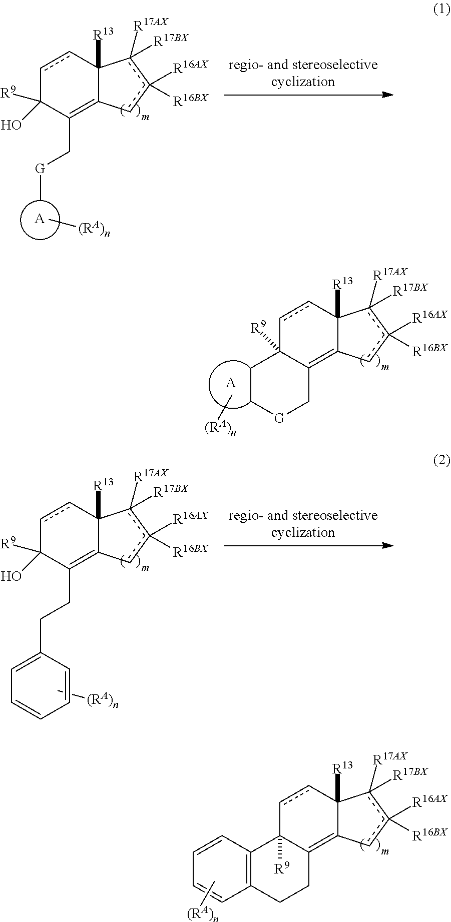 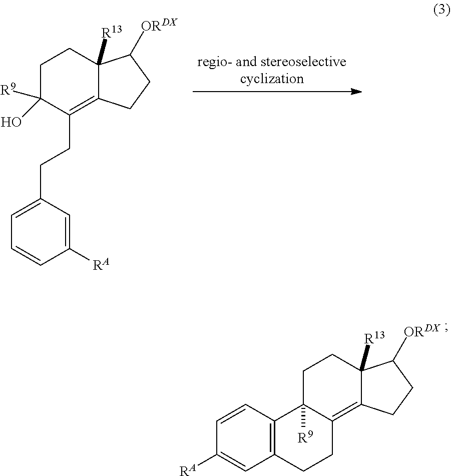
or
(b) a C9-beta-substituted tetracyclic compound (steroid numbering), the method comprising a regio- and stereoselective cyclization reaction depicted in Scheme (4), Scheme (5), or Scheme (6):

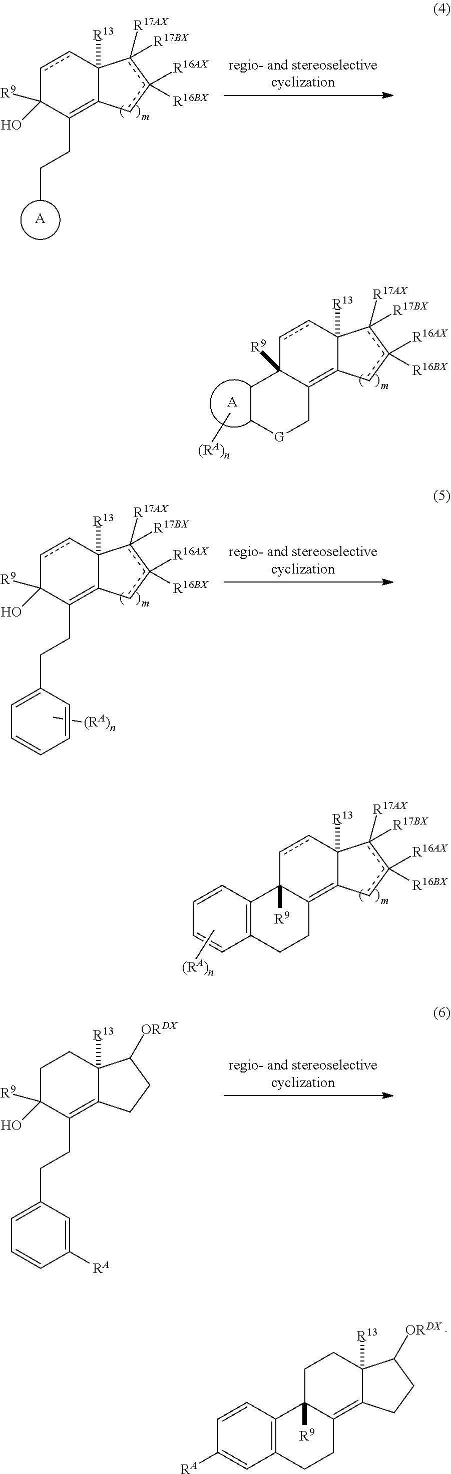

12. A C9-alpha-substituted steroid-like compound or salt or prodrug thereof prepared according to the method of claim 1.

13. A C9-alpha-substituted steroid-like compound or salt or prodrug thereof prepared according to the method of claim 11.

14. A C9-alpha-substituted steroid-like compound or salt or prodrug thereof prepared according to the method of claim 2.

15. A C9-beta-substituted steroid-like compound or salt or prodrug thereof prepared according to the method of claim 1.

16. A C9-beta-substituted steroid-like compound or salt or prodrug thereof prepared according to the method of claim 11.

17. A C9-beta-substituted steroid-like compound or salt or prodrug thereof prepared according to the method of claim 2.

18. The compound or salt or prodrug of claim 12, wherein the C9-alpha-substituted steroid-like compound is a tetracyclic compound.

19. A method for modulating nuclear hormone receptor activity, the method comprising exposing a nuclear hormone receptor to and/or contacting a nuclear hormone with an effective amount of a compound of claim 12 or salt or prodrug thereof.

20. A compound selected from the group consisting of a compound of Formula (Ei), Formula (Fi), Formula (Gi), Formula (Hi), Formula (Ji), Formula (Ki), Formula (Li), and Formula (Mi): 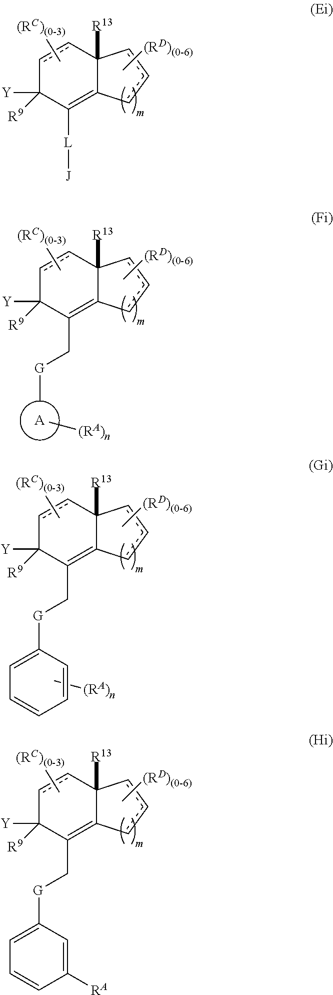 [chemical structure]
